(19) United States
(12) Patent Application Publication
(10) Pub. No.: US 2022/0152118 A1
(43) Pub. Date: May 19, 2022

(54) DIFFERENTIATION METHOD FROM HUMAN ADIPOSE-DERIVED MESENCHYMAL STEM CELLS TO DERMAL PAPILLA CELLS

(71) Applicant: RADIANT.CO.LTD, Chuncheon-si, Gangwon-do (KR)

(72) Inventor: Su Yeon LEE, Chuncheon-si (KR)

(73) Assignee: RADIANT.CO.LTD, Chuncheon-si, Gangwon-do (KR)

(21) Appl. No.: 17/439,045

(22) PCT Filed: Sep. 25, 2020
(86) PCT No.: PCT/KR2020/013108

§ 371 Date: Sep. 14, 2021

(30) Foreign Application Priority Data
Jan. 31, 2020 (KR) 10-2020-0011970

(51) Int. Cl. A61K 35/36; C12N 5/071

(52) U.S. Cl. CPC A61K 35/36; C12N 5/0628; C12N 2501/155; C12N 2533/54; C12N 2501/115; C12N 2506/1384

(57) ABSTRACT

The present invention relates to a method for differentiating from human adipose-derived mesenchymal stem cells to dermal papilla cells using a differentiation induction medium composition in a cell culture plate for inducing differentiation, including gelatin, and to the medium composition. The plate comprising the gelatin of the present invention can exhibit the effect of inducing direct cross-differentiation from human adipose-derived mesenchymal stem cells to dermal papilla cells, and the effect of efficiently enabling mass cultivation ex vivo at low cost by using an economical material. In addition, the dermal papilla cells differentiated from human adipose-derived mesenchymal stem cells of the present invention can be used as a cell therapy composition for preventing or treating hair loss.

### CROSS-REFERENCE TO RELATED APPLICATIONS

This application is a National Stage of International Application No. PCT/KR2020/013108 filed Sep. 25, 2020, which claims priority based on Korean Patent Application No. 10-2020-0011970 filed on Jan. 31, 2020, the entire disclosures of which are incorporated herein by reference.

### TECHNICAL FIELD

The present invention relates to a method for differentiating human adipose-derived mesenchymal stem cells into dermal papilla cells using a differentiation-inducing medium composition on a cell culture plate for inducing differentiation containing gelatin and the differentiation-inducing medium composition.

### BACKGROUND ART

Alopecia (hair loss) may be classified into male pattern hair loss, female pattern hair loss, telogen effluvium, and alopecia areata. Due to various factors, the dermal papilla cells constituting hair follicles interact with the cells near the hair follicles, affect the formation and growth of hair, and function to regulate the growth cycle. Hair follicles degenerate due to various factors, thus inhibiting hair growth and causing hair loss.

Recent statistics from the National Health Insurance Corporation show that 210,000 patients seek treatment for hair loss annually, of which about 44% are in their 20s and 30s, and 45% are female patients, which indicates that hair loss occurs regardless of age or gender. In addition, the proportion of teenage or children patients with hair loss is also gradually increasing, so awareness that hair loss is no longer a problem only for middle-aged and elderly people, but is a disease, is spreading throughout society.

Autologous hair implantation and drug therapy are mainly used to treat hair loss. Autologous hair implantation has an advantage in that treatment is permanent, but has disadvantages in that a high cost is incurred and multiple operations are required when restoring hair over a large hair loss area. In addition, drug therapy is easy to administer and receive, but is only capable of delaying the progress of hair loss or aiding to maintain the current state, rather than being a permanent treatment method, and has a limitation in that side effects are caused by the drug. In addition, several methods, such as gene therapy, have been developed, but the safety and effectiveness thereof have not yet been proven, so it will take time for the methods to be clinically applied.

In recent years, methods for applying stem cells to the treatment of hair loss have been receiving attention. Attempts have been made to realize methods of injecting adipose-derived stem cells into several parts of the scalp and methods including inducing differentiation of adipose-derived stem cells into dermal papilla cells based on the multipotency of stem cells and then forming hair follicles in vivo through implantation. However, the injected adipose-derived stem cells do not enable the formation of new hair follicles, which is the fundamental treatment for hair loss, and have problems in that the stability of cells differentiated through genetic manipulation is low, there is psychological reluctance to genetic manipulation, and economic feasibility is low, which must be resolved. Therefore, there is the need to develop a method for effectively treating hair loss that is safe, economical and effective, and avoids genetic manipulation.

### DISCLOSURE

### Technical Problem

Therefore, the present invention has been made to solve the above problems, and it is one object of the present invention to provide a method for inducing differentiation of human adipose-derived mesenchymal stem cells into dermal papilla cells using a medium composition for inducing differentiation on a cell culture plate for inducing differentiation containing gelatin, which is capable of conveying an economic effect of providing efficient mass culture in vitro, and the medium composition.

### Technical Solution

In accordance with an aspect of the present invention, the above and other objects can be accomplished by the provision of a plate for inducing differentiation into dermal papilla cells, the plate being coated with gelatin.

Meanwhile, the plate is preferably a polystyrene plate.

Meanwhile, the plate preferably aims to induce differentiation of human adipose-derived stem cells into dermal papilla cells.

In accordance with another aspect of the present invention, provided is a method for inducing differentiation of human adipose-derived mesenchymal stem cells into dermal papilla cells, the method including a) adding a medium for culturing animal cells to a plate coated with gelatin, loading human adipose-derived mesenchymal stem cells on the plate, and culturing the human adipose-derived mesenchymal stem cells, (b) culturing the human adipose-derived mesenchymal stem cells cultured in the medium for culturing animal cells in step (a) in a medium for primary differentiation, and (c) culturing the human adipose-derived mesenchymal stem cells cultured in the medium for primary differentiation of step (b) in a medium for secondary differentiation, wherein the medium for primary differentiation in step (b) is prepared by adding retinoic acid, fetal bovine serum (FBS), penicillin, and streptomycin to the medium for culturing animal cells, and the medium for secondary differentiation in step (c) is prepared by adding fibroblast growth factor-2 (bFGF), bone morphogenetic protein 2 (human recombinant BMP2), glycogen synthase kinase 3α/β inhibitor (6-bromoindirubin-3′-oxime), fetal bovine serum (FBS), penicillin, and streptomycin to the medium for culturing animal cells.

In accordance with another aspect of the present invention, provided is a cosmetic composition for promoting hair growth or preventing hair loss, the cosmetic composition containing the dermal papilla cells differentiated from human adipose-derived stem cells by the method for inducing differentiation.

In accordance with another aspect of the present invention, provided is a pharmaceutical composition for promoting hair growth or preventing hair loss, the pharmaceutical composition containing the dermal papilla cells differentiated from human adipose-derived stem cells by the method for inducing differentiation.

The pharmaceutical composition is preferably an external preparation for the skin.

### Advantageous Effects

The plate containing gelatin according to the present invention is capable of exerting an effect of inducing direct cross-differentiation of human adipose-derived mesenchymal stem cells into dermal papilla cells, and is capable of exerting an effect of efficiently performing mass culture in vitro at low cost using economically efficient materials.

In addition, the dermal papilla cells differentiated from human adipose-derived mesenchymal stem cells according to the present invention can be used as a cell therapy composition for preventing or treating hair loss.

### BRIEF DESCRIPTION OF DRAWINGS

FIG. 1 is a graph showing the results of experimentation to determine the gene expression pattern of dermal papilla cell-specific genes with respect to differentiated human adipose-derived mesenchymal stem cells (dADSC) according to the present invention, wherein, for comparison, dermal papilla cells (DPC) and undifferentiated human adipose-derived mesenchymal stem cells (ADSC) were used.

FIG. 2 shows the result of experimentation through flow cytometry (FACS) on dermal papilla cell-specific genes with respect to the differentiated human adipose-derived mesenchymal stem cells (dADSC) according to the present invention, wherein, for comparison, dermal papilla cells (DPC) and undifferentiated human adipose-derived mesenchymal stem cells (ADSC) were used, and wherein a) shows the result of flow cytometry and b) is a graph numerically showing the result thereof.

FIG. 3 shows grouping data (hierarchical clustering & MDS plot) between samples obtained through a microarray.

FIG. 4 shows similar dermal papilla cell gene expression patterns (Wnt signals) between respective cells identified through the microarray.

FIG. 5 is a schematic diagram illustrating a predicted related similar signaling pathway based on the result of microarray assay.

### BEST MODE

The present invention provides a plate for inducing differentiation into dermal papilla cells, wherein the plate is coated with gelatin. The gelatin-coated plate according to the present invention is capable of exerting effects of inducing direct cross-differentiation of human adipose-derived mesenchymal stem cells into dermal papilla cells and of efficiently performing mass culture in vitro at low cost using economically efficient materials.

Meanwhile, in the present invention, the plate is preferably a polystyrene plate.

Meanwhile, in the present invention, the plate should use an appropriate extracellular matrix molecule for the differentiation and proliferation of stem cells. Examples of useful extracellular matrix molecules include collagen, fibronectin, Matrigel, gelatin, and the like. In the present invention, gelatin, which has the highest cell differentiation and proliferation rates, is preferably used, and a culture plate which is coated with gelatin while excluding feeder cells is used. Based thereon, it is possible to obtain effects of obviating a co-culture step for proliferation and differentiation, thus reducing the time required for differentiation and enabling rapid application to patients, and of reducing the possibility of disease transmission due to contamination in the process of culturing feeder cells, thereby improving the stability of differentiated cells.

Meanwhile, in the present invention, the gelatin contained in the plate may contain gelatin at various concentrations, preferably 0.1 to 0.2% by weight.

Meanwhile, in the present invention, the plate preferably aims to induce differentiation of human adipose-derived stem cells into dermal papilla cells.

The present invention provides a method for inducing differentiation of human adipose-derived mesenchymal stem cells into dermal papilla cells, the method including: a) adding a medium for culturing animal cells to a plate coated with gelatin, loading human adipose-derived mesenchymal stem cells on the plate, and culturing the human adipose-derived mesenchymal stem cells, (b) culturing the human adipose-derived mesenchymal stem cells cultured in the medium for culturing animal cells in step (a) in a medium for primary differentiation, and (c) culturing the human adipose-derived mesenchymal stem cells cultured in the medium for primary differentiation of step (b) in a medium for secondary differentiation, wherein the medium for primary differentiation in step (b) is prepared by adding retinoic acid, fetal bovine serum (FBS), penicillin, and streptomycin to the medium for culturing animal cells, and the medium for secondary differentiation in step (c) is prepared by adding fibroblast growth factor-2 (bFGF), bone morphogenetic protein 2 (human recombinant BMP2), glycogen synthase kinase 3α/β inhibitor (6-bromoindirubin-3′-oxime), fetal bovine serum (FBS), penicillin, and streptomycin to the medium for culturing animal cells.

In the method for inducing differentiation of human adipose-derived stem cells into dermal papilla cells of the present invention, the human adipose-derived stem cells are loaded on a plate coated with gelatin, and then differentiation of dermal papilla cells is induced sequentially using a medium for culturing animal cells, a medium for primary differentiation, and a medium for secondary differentiation. Any medium for culturing animal cells may be used, as long as it is one that is used for culturing animal cells in the art. However, the medium preferably contains fetal bovine serum (FBS), penicillin, and streptomycin, and more preferably is a commercially available DMEM/high-glucose medium supplemented with fetal bovine serum (FBS), penicillin and streptomycin.

Meanwhile, in the medium for culturing animal cells according to the present invention, the fetal bovine serum is preferably present in an amount of 5 to 15%, more preferably 10%.

In addition, in the medium for primary differentiation according to the present invention, the retinoic acid is preferably present in an amount of 0.001 to 1 mM, more preferably 0.001 to 0.1 mM.

In addition, in the medium for secondary differentiation according to the present invention, the fibroblast growth factor-2 is preferably present in an amount of 1 to 1,000 ng/ml, more preferably 1 to 40 ng/ml. In addition, in the medium for secondary differentiation according to the present invention, the bone morphogenetic protein 2 is preferably present in an amount of 1 to 1,000 ng/ml, more preferably 1 to 400 ng/ml. In addition, in the medium for secondary differentiation according to the present invention, the glycogen synthase kinase 3α/β inhibitor is preferably present in an amount of 1 to 100 μM, more preferably 1 to 10 μM.

Meanwhile, in the method for inducing differentiation according to the present invention, in steps (a) to (c), the culture is preferably carried out in the presence of 3 to 7% CO₂ at a temperature of 35 to 39° C., specifically in the presence of 5% CO₂ at a temperature of 37° C., in the present invention. At this time, the optimal temperature for cell culture is a requirement that mainly depends on the body temperature of the host from which the cells are isolated, and 5% CO₂ is a requirement for preventing CO₂ generated in the medium during cell metabolism from being vaporized into an incubator for the normal action of phenol red, which is a pH indicator added to determine the time of nutrient consumption in the nutrient-limited medium to monitor cell metabolism and growth.

Meanwhile, in the method for inducing differentiation according to the present invention, in step (a), cell adhesion is stabilized and culture is preferably performed for 1 to 2 days, specifically, for 1 day in the present invention. In addition, in step (b), treatment with a cell differentiation promoter is performed, and culture is preferably performed for 1 to 7 days, specifically for 3 days, in the present invention. In addition, in step (c), treatment with a factor for inducing dermal papilla cell characteristics is performed, and the culture is preferably performed for 1 to 14 days, specifically for 3 days, in the present invention.

Meanwhile, in the method of inducing differentiation according to the present invention, the medium used in steps (b) and (c) was replaced once every day. This aims to maintain the freshness of the medium. When factors treated along with the differentiation medium are maintained at a high temperature for a long time, the activity thereof decreases. For this reason, the medium is preferably frequently replaced.

Meanwhile, in the present invention, the human adipose-derived stem cells are preferably human adipose-derived mesenchymal stem cells. The mesenchymal stem cells are preferably derived from bone marrow, adipose tissue, or umbilical cord.

Meanwhile, in previous related studies, cells of different origins were induced into IPS cells and then differentiated. However, the method for inducing differentiation of human adipose-derived stem cells into dermal papilla cells according to the present invention is characterized in that mesenchymal stem cells are directly converted (trans-differentiated) into dermal papilla cells.

Meanwhile, in the present invention, the mesenchymal stem cells differentiated into dermal papilla cells preferably express any one or more selected from the group consisting of LEF-1, Corin, and Wnt5a, which are dermal papilla cell-specific genes.

The present invention provides a cosmetic composition for promoting hair growth or preventing hair loss, the cosmetic composition containing the dermal papilla cells differentiated from human adipose-derived stem cells by the method for inducing differentiation.

Meanwhile, for example, the cosmetic composition of the present invention may be any one formulation selected from hair serum, hair tonic, hair essence, hair treatment, hair shampoo, hair conditioner, hair lotion, and scalp/hair treatment. Any formulation may be used without particular limitation, as long as it is commonly used in the field of cosmetics for the scalp and hair, and can be selected and combined without difficulty by those skilled in the art depending on the type or purpose of use of other external agents.

Meanwhile, the cosmetic composition according to the present invention may contain one or more adjuvants commonly used in the cosmetics field, for example, hydrophilic or lipophilic active agents, preservatives, antioxidants, solvents, fragrances, fillers, blockers, pigments, deodorants, dyes, and the like. These various adjuvants are present in amounts conventionally used in the art, for example, 0.001 to 30% by weight with respect to the total weight of the composition. However, in any case, the adjuvants and the ratio thereof will be selected so as not to adversely affect the desirable properties of the cosmetic composition according to the present invention.

Meanwhile, the cosmetic composition of the present invention may be used in combination with a cosmetic composition other than the cosmetic composition according to the present invention. In addition, the cosmetic composition according to the present invention may be used according to a conventional method of use, and the number of applications may vary depending on the skin condition or preference of the user.

The present invention provides a pharmaceutical composition for promoting hair growth or preventing hair loss, the pharmaceutical composition containing the dermal papilla cells differentiated from human adipose-derived stem cells by the method for inducing differentiation.

Meanwhile, the pharmaceutical composition of the present invention may be, for example, provided as an oral formulation, an external preparation for the skin, a suppository, or a sterile injectable solution, and is preferably an external preparation for the skin.

Meanwhile, in the pharmaceutical composition of the present invention, when the oral formulation is a solid preparation, it may, for example, be a tablet, a pill, a powder, a granule, or a capsule. In addition, when the oral formulation is a liquid preparation, it may, for example, be a suspension, an oral solution, an emulsion, or a syrup.

Meanwhile, in the pharmaceutical composition of the present invention, the external preparation for the skin may be prepared as a formulation such as a liquid, cream, paste, or solid.

Meanwhile, for example, the pharmaceutical composition of the present invention is preferably administered in a daily dose of 0.00001 to 100 mg/kg (body weight), but the present invention is not necessarily limited thereto, and preferably, the dose is determined in consideration of the administration method, the age, gender, and weight of the user, and the severity of the disease.

Meanwhile, the pharmaceutical composition of the present invention may further contain a pharmaceutically acceptable carrier, diluent, or excipient, in addition to the active ingredient. Examples of useful carriers, excipients or diluents include one or more of lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. In addition, fillers, anti-aggregants, lubricants, wetting agents, fragrances, emulsifiers, preservatives or the like may be additionally contained when the prophylactic and therapeutic agents are pharmaceuticals.

Meanwhile, according to the following experiment, the differentiated human adipose-derived mesenchymal stem cells according to the present invention exhibited an increase in the gene expression patterns of LEF-1, Corin, and Wnt5a, which are specific genes of dermal papilla cells, compared to wild human adipose-derived mesenchymal stem cells. In addition, flow cytometry showed that the proportion of cells that are positive for dermal papilla cell-specific genes, namely, LEF-1, Corin, and Wnt5a, was more than 90%. This result was similar to that for dermal papilla cells. In addition, the result of analysis through microarray and qPCR to determine the similarity between the differentiated human adipose-derived mesenchymal stem cells according to the present invention and dermal papilla cells showed that the differentiated human adipose-derived mesenchymal stem cells according to the present invention, like dermal papilla cells, activate Wnt signaling, which is a representative signaling pathway of the dermal papilla cells, which means that the differentiated human adipose-derived mesenchymal stem cells have similar signaling to the dermal papilla cells.

Therefore, the present invention can provide an alternative material for dermal papilla cells that is capable of exerting an effect of efficiently enabling mass culture in vitro at low cost based on the differentiation medium composition for direct cross-differentiation of human adipose-derived mesenchymal stem cells into dermal papilla cells and the differentiation method using the same. In addition, the present invention can provide a potent cell therapy composition for preventing and treating hair loss using the alternative material.

Hereinafter, the present invention will be described in more detail with reference to the following examples and experimental examples, but the scope of the present invention is not limited thereto, and includes variations and technical concepts equivalent thereto.

### Example 1: Differentiation of Human Adipose-Derived Mesenchymal Stem Cells into Dermal Papilla Cells and Characterization

In this example, differentiation of human adipose-derived mesenchymal stem cells into dermal papilla cells was induced, and the characteristics of the dermal papilla cells were determined.

1) Induction of Differentiation of Human Adipose-Derived Mesenchymal Stem Cells into Dermal Papilla Cells

Human adipose-derived mesenchymal stem cells were transferred to a conventional 6-well plate (CoStar) and to a gelatin-coated polystyrene (PS) plate (6-well clear TC-treated multiple well plate, 3516, Costar, Corning, N.Y., USA) (1×10⁵ cells per well). The medium used herein was DMEM/high-glucose medium [(Dulbecco's modified Eagle's medium—high-glucose liquid medium (SH30243, HyClone, UT, USA) supplemented with 10% fetal bovine serum (FBS) and 1×penicillin/streptomycin, the detailed medium composition being as shown in Table 1 below], and the cells were cultured in the presence of 5% CO₂ at a temperature of 37° C. for 1 day.

TABLE 1

| | mg/L | mmol/L |
|---|---|---|
| Inorganic salts | | |
| Calcium chloride | 200 | 1.8021 |
| Ferric nitrate-9H₂0 | 0.1 | 0.0002 |
| Potassium chloride | 400 | 5.3655 |
| Magnesium sulfate | 97.67 | 0.8112 |
| Sodium chloride | 6400 | 109.514 |
| Sodium phosphate | 125 | 0.9059 |
| monobasic H₂0 | | |
| Amino acids | | |
| L-Arginine-HCl | 84 | 0.3987 |
| L-Cystine-2HCl | 62.57 | 0.1998 |
| L-Glutamine | 584 | 3.9959 |
| Glycine | 30 | 0.3996 |
| L-Histidine-HCl-H₂0 | 42 | 0.2004 |
| L-Isoleucine | 104.8 | 0.7989 |
| L-Leucine | 104.8 | 0.799 |
| L-Lysine-HCl | 146.2 | 0.8004 |
| L-Methionine | 30 | 0.2011 |
| L-Phenylalanine | 66 | 0.3995 |
| L-Serine | 42 | 0.3997 |
| L-Threonine | 95.2 | 0.7992 |
| L-Tryptophan | 16 | 0.0783 |
| L-Tyrosine-2Na-2H₂0 | 103.79 | 0.3974 |
| L-Valine | 93.6 | 0.799 |
| Vitamins | | |
| Calcium D-pantothenate | 4 | 0.0084 |
| D-Pantothenic acid Na salt | 0 | 0 |
| Choline chloride | 4 | 0.0286 |
| Folic acid | 4 | 0.0091 |
| Myo-inositol | 7 | 0.0389 |
| Niacinamide | 4 | 0.0328 |
| Pyridoxine-HCl | 4 | 0.0195 |
| Riboflavin | 0.4 | 0.0011 |
| Thiamine-HCl | 4 | 0.0119 |
| Other | | |
| D-Glucose | 4500 | 24.9778 |
| HEPES | 0 | 0 |
| Phenol red-Na | 15.9 | 0.0422 |
| Sodium pyruvate | 110 | 0.9996 |
| Sodium bicarbonate | 3700 | 44.0424 |

To induce differentiation of cells cultured on conventional and gelatin-coated plates into dermal papilla cells, the DMEM/high-glucose differentiation medium supplemented with 0.01 mM retinoic acid, 10% fetal bovine serum and 1×penicillin/streptomycin was incubated in an amount of 2 ml per a 6-well plate at 5% CO₂ and a temperature of 37° C. for 3 days. At this time, the medium was replaced with fresh medium once every day for 3 days. The existing culture medium was removed (by suction) and 2 ml of fresh DMEM/high-glucose differentiation medium was then provided using a pipette.

Then, 2 ml of the DMEM/high-glucose differentiation medium supplemented with 20 ng/ml fibroblast growth factor-2 (bFGF), 200 ng/ml bone morphogenetic protein 2 (human recombinant BMP2), 1 μM glycogen synthase kinase 3α/β inhibitor (6-bromoindirubin-3′-oxime), 10% fetal bovine serum, and 1×penicillin/streptomycin was incubated in an amount of 2 ml per 6-well plate at 5% CO₂ and a temperature of 37° C. for 4 days. At this time, the medium was replaced with fresh medium once every day for 4 days. The existing culture medium was removed (by suction), 2 ml of fresh DMEM/high-glucose differentiation medium was then provided using a pipette to induce differentiation.

2) Characterization of Human Adipose-Derived Mesenchymal Stem Call-Derived Dermal Papilla Cells Based on Gene Expression Pattern

In order to determine whether or not the characteristics of human adipose-derived mesenchymal stem cells differentiated according to the method described above were similar to those of dermal papilla cells, the gene expression patterns of LEF-1, Corin, and Wnt5a, which are dermal papilla cell-specific genes, were verified.

Total RNA was isolated from wild human adipose-derived mesenchymal stem cells, dermal papilla cells, and differentiated human adipose-derived mesenchymal stem cells using chloroform and isopropanol. cDNA was synthesized using a Maxima First Strand cDNA Synthesis Kit (Thermo Fisher) and the RNA as a template. Then, quantitative reverse transcription polymerase chain reaction analysis was performed using EmeraldAmp® GT PCR Master Mix (Takara Bio), and the primer sequences used herein are shown in Table 2 below.

TABLE 2

| | | | Sequence |
|---|---|---|---|
| Gene | Direction | Sequence (5′-3′) | Number |
| LEF-1 | Forward | GAGAGCGAATGTCGTTCGTG | SEQ NO: 1 |
| | Reverse | GGGTGCTGATGGATAGCTGGT | SEQ NO: 2 |
| Corin | Forward | GTCATTTCAAGTGCCGCTCA | SEQ NO: 3 |
| | Reverse | GGTTTGCACATTCCAGCTCA | SEQ NO: 4 |
| Wnt5a | Forward | TGAACCTGCACAACAACGAG | SEQ NO: 5 |
| | Reverse | TGACCTGTACCAACTTGCCC | SEQ NO: 6 |
| β-actin | Forward | GAGCACAGAGCCTCGCCTTT | SEQ NO: 7 |
| | Reverse | AGAGGCGTACAGGGATAGCA | SEQ NO: 8 |

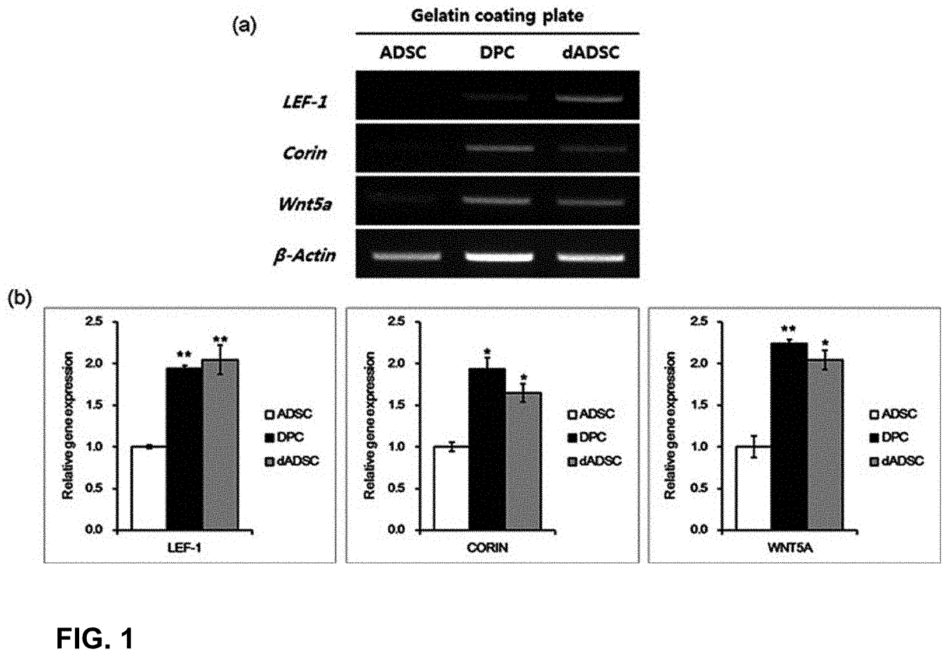

As a result, as can be seen from FIG. 1, the gene expression patterns of LEF-1, Corin, and Wnt5a, which are dermal papilla cell-specific genes, were increased in dermal papilla cells (DPC) and differentiated human adipose-derived mesenchymal stem cells (dADSC) obtained by the method described above, compared to human adipose-derived mesenchymal stem cells (ADSC).

3) Characterization of Human Adipose-Derived Mesenchymal Stem Cell-Derived Dermal Papilla Cells Through Flow Cytometry (FACS)

The differentiation potential of the differentiated human adipose-derived mesenchymal stem cells according to the method was determined based on LEF-1, Corin, and Wnt5a, which are dermal papilla cell-specific genes.

The differentiated human adipose-derived mesenchymal stem cells (dADSC) and dermal papilla cells (DPC) were removed by treatment with 0.05% trypsin/0.02% EDTA, the concentration thereof was then adjusted to 2×10⁵ cells/ml, and the cell solution was blocked with Fc receptors. Then, the cells were fixed using a fixation solution of a fixation/permeabilization solution kit (BD Cytofix/Cytoperm™), and LEF-1, Corin, and Wnt5a, which are dermal papilla cell-specific genes, were stained with a permeabilization solution. The stained cells were washed with a staining solution, suspended in fresh staining solution, and then analyzed with a flow cytometer (FACSCalibur, BD science) and CellQuest software (CELLQUEST software; BD science).

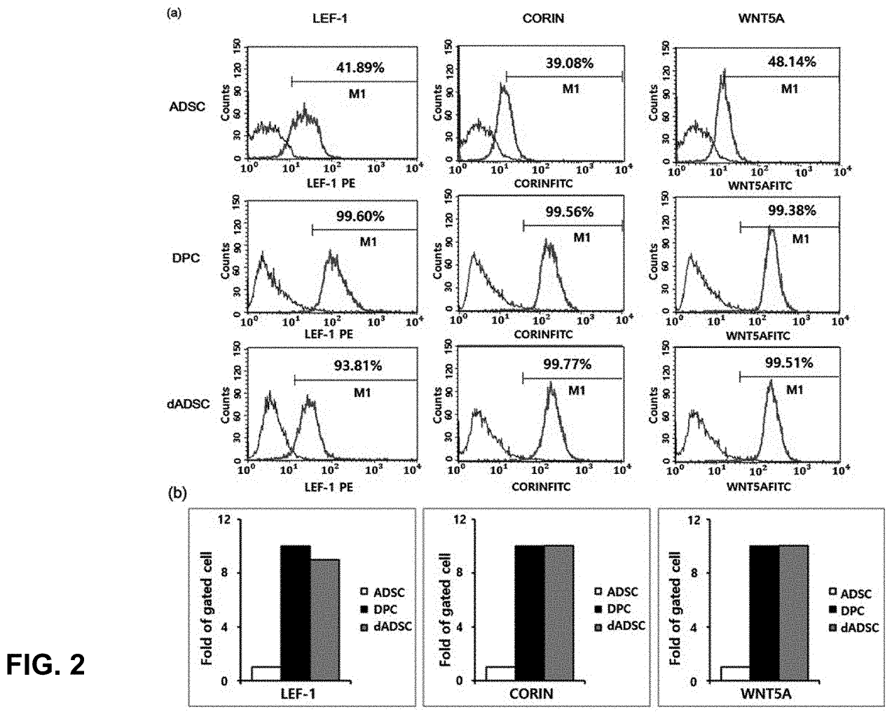

As a result, as can be seen from FIG. 2, the differentiated human adipose-derived mesenchymal stem cells (dADSC) were found to have a percentage of cells positive to Corin, Wnt5a, and LEF-1, which are dermal papilla cell-specific genes, of 90% or more compared to that of wild human adipose-derived mesenchymal stem cells (ADSC), which was similar to that for dermal papilla cells.

### Experimental Example 1: Determination of Similarity Between Cells Differentiated from Human Adipose-Derived Mesenchymal Stem Cells and Dermal Papilla Cells

In this experimental example, in order to determine the similarity between dermal papilla cells and differentiated human adipose-derived mesenchymal stem cells according to the method above, genomic profiling was primarily performed based on a database of cells obtained using a microarray to screen significant genes, and then signaling similar to that of the target cells, that is, the dermal papilla cells, was finally identified through qPCR (real-time PCR) verification.

A microarray is a tool that can be used to measure the amount of expression of all or some of the genes of an organism, and is capable of obtaining various results through the establishment of an integrated database containing biological information of cells. Therefore, the database of the differentiated human adipose-derived mesenchymal stem cells was established by identifying the expression patterns between dermal papilla cell genes and differentiated human adipose-derived mesenchymal stem cell genes of the present invention through the present method.

RNA was isolated from the differentiated human adipose-derived mesenchymal stem cells, wild human adipose-derived mesenchymal stem cells, and dermal papilla cells, a microarray assay (genome U133 plus 2.0 chip from Affymetrix Corp.) was performed on samples verified through the RNA quality control, and the results were scanned using a GCS3000 Scanner (Affymetrix Corp.). After scanning, the resulting values were extracted by RMA analysis (background correction, summarization, normalization) using Affymetrix Power Tools (APT) Software. The test conditions were as shown in Table 3.

TABLE 3

| Sample type | Total RNA |
|---|---|
| Platform | GeneChip ® Human Gene 2.0 ST Array |
| cDNA | cDNA was synthesized using the GeneChip WT |
| synthesize | (Whole Transcript) Amplification kit as described |
| | by the manufacturer |
| Label protocol | the sense cDNA was then fragmented and biotin- |
| | labeled with TdT (terminal deoxynucleotidyl |
| | transferase) using the GeneChip WT |
| | Terminal labeling kit |
| Hybridization | Approximately 5.5 mg of labeled DNA target |
| protocol | was hybridized to the Affymetrix GeneChip |
| | Array at 45° C. for 16 hours |
| Scan protocol | Hybridized arrays were washed and stained on a |
| | GeneChip Fluidics Station 450 and scanned on a |
| | GCS3000 Scanner (Affymetrix) |
| Data | Array data export processing and analysis was |
| processing | performed using Affymetrix ® GeneChip |
| | Command Console ® Software (AGCC) |
| Software | Affymetrix Power Tools (affymetrix-power- |
| | tools.html) R 3.5.1 (http://www.r-project.org/) |

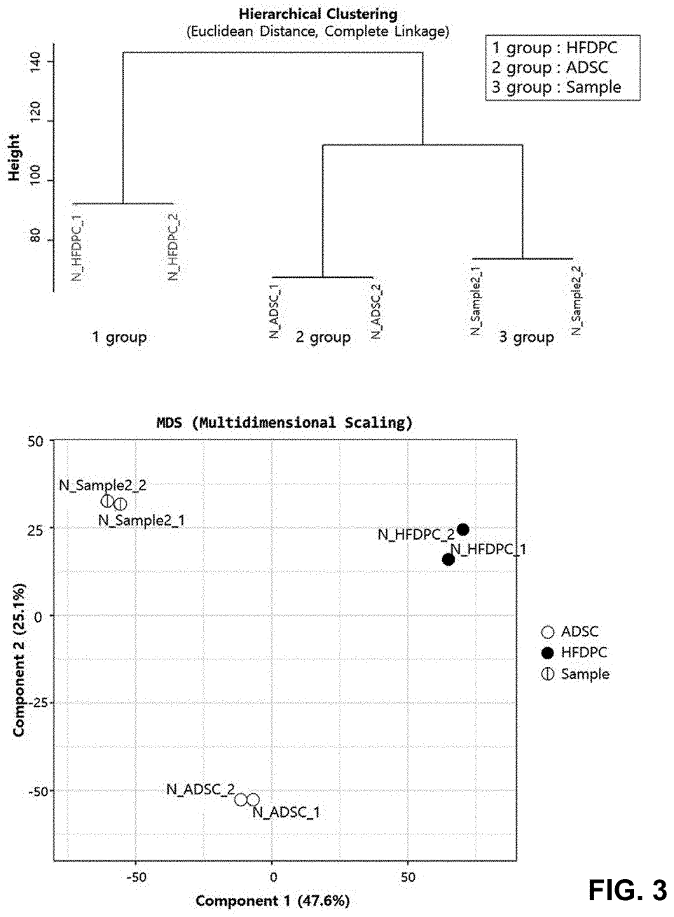

A group of genes of interest, that is, a group of similarly expressed genes, was derived from dermal papilla cells (HFDPC) and differentiated human adipose-derived mesenchymal stem cells through the microarray as described above. As a result, as can be seen from FIG. 3, the differentiated human adipose-derived mesenchymal stem cells (Sample) were classified into a different group from the wild human adipose-derived mesenchymal stem cells (ADSC), but included a part of the average linkage. Thus, this indicates that the differentiated human adipose-derived mesenchymal stem cells were transformed into cells having characteristics different from those of the wild human adipose-derived mesenchymal stem cells.

In addition, in order to determine the change and similarity of the expression pattern for each cell, genetic analysis was performed on a total of 53,617 genes by applying “the result of GO/KEGG analysis on the probe list that did not satisfy the cut-off in the result for HFDPC vs Sample but satisfied the cut-off in the result for ADSC vs Sample (cut-off: |fc|≥2 & lpe.p<0.05)” to the genes. The result identified 85 genes having expression patterns similar to dermal papilla cells and 21 similar gene-related signaling. Among them, “Wnt signaling pathway” had the greatest concentration of related genes, and is signaling associated with hair differentiation and regeneration. Wnt signaling is known to play an important role in processes such as activation of hair follicle stem cells, which is essential for hair growth and regeneration, and proliferation of hair germ cells, and the corresponding signaling is also known to be involved in the mechanism of differentiation into dermal papilla cells.

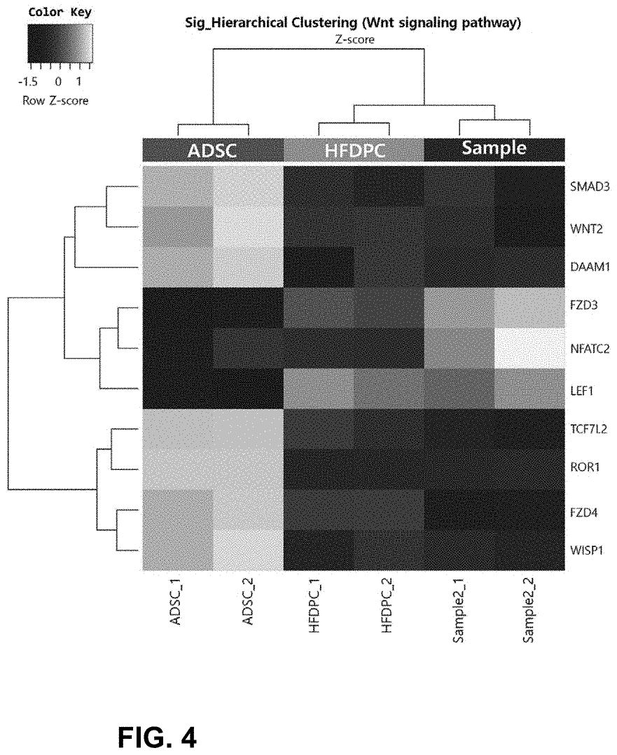

For the genes identified to be similar to dermal papilla cells, the factors related to Wnt signaling are SMAD3, LEF1, WISP1, ROR1, DAAM1, TCF7L2, WNT2, FZD4, NFATC2, and FZD3. When the difference in gene expression between wild human adipose-derived mesenchymal stem cells (ADSC), dermal papilla cells (HFDPC), and the differentiated human adipose-derived mesenchymal stem cells (Sample) was determined, the gene expression patterns between the dermal papilla cells and differentiated human adipose-derived mesenchymal stem cells were similar, as shown in FIG. 4. Based on these results, the hypothesis that the differentiated human adipose-derived mesenchymal stem cells could activate Wnt signaling in a manner similar to dermal papilla cells was proposed and verified. Therefore, the predicted similar signaling and differentiation mechanism pathways including the similarly expressed genes identified based on the results of microarray, and the genes identified to be dominant in the differentiated human adipose-derived mesenchymal stem cells or dermal papilla cells than in the wild human adipose-derived mesenchymal stem cells were established, genes related to each mechanism were selected, mechanisms similar to those of dermal papilla cells were determined through qPCR, and the results of the microarray assay were verified.

Total RNA was isolated from wild human adipose-derived mesenchymal stem cells, dermal papilla cells, and differentiated human adipose-derived mesenchymal stem cells using chloroform and isopropanol. cDNA was synthesized using the RNA as a template and using a Maxima First Strand cDNA Synthesis Kit (Thermo Fisher). Then, quantitative reverse transcription polymerase chain reaction (qPCR) analysis was performed using LightCycler 480 SYBR Green I Master (2× conc.) (Roche), and the primer sequences used herein were as shown in Table 4.

TABLE 4

| | | | Sequence |
|---|---|---|---|
| Gene | Direction | Sequence (5′-3′) | Number |
| FZD3 | Forward | CAGGGTCCTAGTGGAGGATGT | SEQ NO: 9 |
| | Reverse | AGAGGATCAGCAGTGCCACG | SEQ NO: 10 |
| BAMBI | Forward | CGCCACTCCAGCTACATCTT | SEQ NO: 11 |
| | Reverse | CAGTGGGCAGCATCACAGTA | SEQ NO: 12 |
| TCF7 | Forward | CACCACACTCCCTGTCCAAG | SEQ NO: 13 |
| | Reverse | CTGGGCCAGTTTGTCTCTGAT | SEQ NO: 14 |
| PLCB4 | Forward | GGAACAGAGGACACTGAGGAC | SEQ NO: 15 |
| | Reverse | TTCAGGTCCTACTATGGAGAGTG | SEQ NO: 16 |
| Wnt5a | Forward | CCAGCTCTGCCCCAACTC | SEQ NO: 17 |
| | Reverse | CGGAGCGACCGGGTTAAG | SEQ NO: 18 |
| LEF-1 | Forward | AGAGCATCTTGCATCCAAACCT | SEQ NO: 19 |
| | Reverse | GGGTGCTGATGGATAGCTGGTT | SEQ NO: 20 |
| β-actin | Forward | CTTCGCGGGCGACGAT | SEQ NO: 21 |
| | Reverse | ATAGGAATCCTTCTGACCCATGC | SEQ NO: 22 |

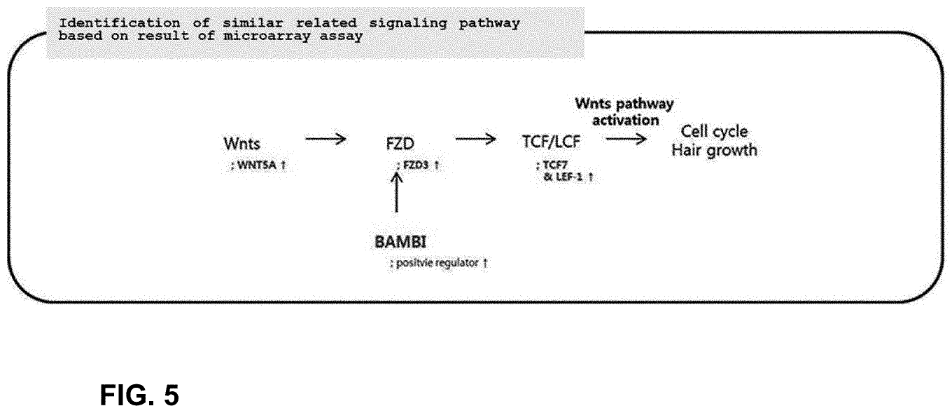

As a result, as can be seen from Table 5 and FIG. 5, the expression patterns of FZD3, BAMBI, TCF7, PLCB4, Wnt5a, and LEF-1, which are target genes for similar signaling, were similar to the microarray results, and the gene expression of dermal papilla cells and the differentiated human adipose-derived mesenchymal stem cells was increased compared to that for wild human adipose-derived mesenchymal stem cells. This proved that Wnt signaling, which is related to hair regeneration and growth and is the representative signaling of dermal papilla cells, can be activated in the differentiated human adipose-derived mesenchymal stem cells, which supported the notion that the differentiated human adipose-derived mesenchymal stem cells have signaling similar to dermal papilla cells.

TABLE 5

| | | | | | | LEF- |
|---|---|---|---|---|---|---|
| | FZD3 | BAMBI | TCF7 | PLCB4 | Wnt5a | 1 |
| Microarray | | | | | | |
| Human adipose-derived | 1.0 | 1.0 | 1.0 | 1.0 | 1.0 | 1.0 |
| mesenchymal stem cells | | | | | | |
| Dermal papilla cells | 2.2 | 2.0 | 1.6 | 57.7 | 8.1 | 3.3 |
| Differentiated human | 4.2 | 9.7 | 2.6 | 13.9 | 3.0 | 3.1 |
| adipose-derived | | | | | | |
| mesenchymal stem cells | | | | | | |
| qPCR | | | | | | |
| Human adipose-derived | 1.0 | 1.0 | 1.0 | 1.0 | 1.0 | 1.0 |
| mesenchymal stem cells | | | | | | |
| Dermal papilla cells | 4.9. | 1.4 | 1.6 | 3.0 | 7.5 | 34.4 |
| Differentiated human | 5.4 | 21.0 | 2.5 | 1.8 | 7.1 | 28 |
| adipose-derived | | | | | | |
| mesenchymal stem cells | | | | | | |

1. A plate for inducing differentiation into dermal papilla cells, the plate being coated with gelatin.

2. The plate according to claim 1, wherein the plate is a polystyrene plate.

3. The plate according to claim 1, wherein the plate aims to induce differentiation of human adipose-derived stem cells into dermal papilla cells.

4. A method for inducing differentiation of human adipose-derived mesenchymal stem cells into dermal papilla cells, the method comprising:
a) adding a medium for culturing animal cells to a plate coated with gelatin, loading human adipose-derived mesenchymal stem cells on the plate, and culturing the human adipose-derived mesenchymal stem cells;
(b) culturing the human adipose-derived mesenchymal stem cells cultured in the medium for culturing animal cells in step (a) in a medium for primary differentiation; and
(c) culturing the human adipose-derived mesenchymal stem cells cultured in the medium for primary differentiation of step (b) in a medium for secondary differentiation,
wherein the medium for primary differentiation in step (b) is prepared by adding retinoic acid, fetal bovine serum (FBS), penicillin, and streptomycin to the medium for culturing animal cells, and
the medium for secondary differentiation in step (c) is prepared by adding fibroblast growth factor-2 (bFGF), bone morphogenetic protein 2 (human recombinant BMP2), glycogen synthase kinase 3α/β inhibitor (6-bromoindirubin-3′-oxime), fetal bovine serum (FBS), penicillin, and streptomycin to the medium for culturing animal cells.

5. A cosmetic composition for promoting hair growth or preventing hair loss, the cosmetic composition comprising the dermal papilla cells differentiated from human adipose-derived stem cells by the method according to claim 4.

6. A pharmaceutical composition for promoting hair growth or preventing hair loss, the pharmaceutical composition comprising the dermal papilla cells differentiated from human adipose-derived stem cells by the method according to claim 4.

7. The pharmaceutical composition according to claim 6, wherein the pharmaceutical composition is an external preparation for skin.

8. A method for promoting hair growth or preventing hair loss, the method comprising administering a composition comprising the dermal papilla cells differentiated from human adipose-derived stem cells by the method according to claim 4 to a subject in need thereof.

9. The method of claim 8, wherein the composition is administered to a skin of the subject.


SEQUENCE LISTING (22 sequences)

SEQ ID NO: 1 - DNA, 20 bp, Artificial Sequence
  LEF-1 Forward (for RT-PCR)
gagagcgaatgtcgttcgtg

SEQ ID NO: 2 - DNA, 21 bp, Artificial Sequence
  LEF-1 Reverse (for RT-PCR)
gggtgctgatggatagctggt

SEQ ID NO: 3 - DNA, 20 bp, Artificial Sequence
  Corin Forward
gtcatttcaagtgccgctca

SEQ ID NO: 4 - DNA, 20 bp, Artificial Sequence
  Corin Reverse
ggtttgcacattccagctca

SEQ ID NO: 5 - DNA, 20 bp, Artificial Sequence
  Wnt5a Forward (for RT-PCR)
tgaacctgcacaacaacgag

SEQ ID NO: 6 - DNA, 20 bp, Artificial Sequence
  Wnt5a Reverse (for RT-PCR)
tgacctgtaccaacttgccc

SEQ ID NO: 7 - DNA, 20 bp, Artificial Sequence
  beta-actin Forward (for RT-PCR)
gagcacagagcctcgccttt

SEQ ID NO: 8 - DNA, 20 bp, Artificial Sequence
  beta-actin Reverse (for RT-PCR)
agaggcgtacagggatagca

SEQ ID NO: 9 - DNA, 21 bp, Artificial Sequence
  FZD3 Forward
cagggtcctagtggaggatgt

SEQ ID NO: 10 - DNA, 20 bp, Artificial Sequence
  FZD3 Reverse
agaggatcagcagtgccacg

SEQ ID NO: 11 - DNA, 20 bp, Artificial Sequence
  BAMBI Forward
cgccactccagctacatctt

SEQ ID NO: 12 - DNA, 20 bp, Artificial Sequence
  BAMBI Reverse
cagtgggcagcatcacagta

SEQ ID NO: 13 - DNA, 20 bp, Artificial Sequence
  TCF7 Forward
caccacactccctgtccaag

SEQ ID NO: 14 - DNA, 21 bp, Artificial Sequence
  TCF7 Reverse
ctgggccagtttgtctctgat

SEQ ID NO: 15 - DNA, 21 bp, Artificial Sequence
  PLCB4 Forward
ggaacagaggacactgaggac

SEQ ID NO: 16 - DNA, 23 bp, Artificial Sequence
  PLCB4 Reverse
ttcaggtcctactatggagagtg

SEQ ID NO: 17 - DNA, 18 bp, Artificial Sequence
  Wnt5a Forward (for qPCR)
ccagctctgccccaactc

SEQ ID NO: 18 - DNA, 18 bp, Artificial Sequence
  Wnt5a Reverse (for qPCR)
cggagcgaccgggttaag

SEQ ID NO: 19 - DNA, 22 bp, Artificial Sequence
  LEF-1 Forward (for qPCR)
agagcatcttgcatccaaacct

SEQ ID NO: 20 - DNA, 22 bp, Artificial Sequence
  LEF-1 Reverse (for qPCR)
gggtgctgatggatagctggtt

SEQ ID NO: 21 - DNA, 16 bp, Artificial Sequence
  beta-actin Forward (for qPCR)
cttcgcgggcgacgat

SEQ ID NO: 22 - DNA, 23 bp, Artificial Sequence
  beta-actin Reverse (for qPCR)
ataggaatccttctgacccatgc